(19) United States
(12) Patent Application Publication
(10) Pub. No.: US 2004/0202614 A1
(43) Pub. Date: Oct. 14, 2004

(54) Utensil spray composition and portable aerosol device and method therefor

(76) Inventors: Roger Davis, Scottsdale, AZ (US); Carrie A. Martz, Scottsdale, AZ (US); Jeremy P. Jones, Phoenix, AZ (US)

Correspondence Address: WEISS & MOY PC, 4204 NORTH BROWN AVENUE, SCOTTSDALE, AZ, 85251, US

(21) Appl. No.: 10/794,378

(22) Filed: Mar. 5, 2004

Related U.S. Application Data
(60) Provisional application No. 60/453,266, filed on Mar. 7, 2003.

(51) Int. Cl. A61K 7/32; A61L 9/04

(52) U.S. Cl. 424/45

(57) ABSTRACT

An anti-bacterial spray composition for utensils and other objects comprises at least one non-toxic anti-bacterial substantially non-residue-producing polar, protic solvent having a low molecular weight and a high level of volatility and at least one residue-producing non-toxic anti-bacterial component, the solvent and the component providing both immediate and long-acting sanitization when applied to a surface of a contaminated object. The anti-bacterial spray composition comprises a combination of Ethanol and Triclosan. A portable aerosol-type dispenser comprising the anti-bacterial spray composition is used by a person to disinfect utensils and objects so that the user's mouth coming into both direct and indirect contact with the anti-bacterial composition is unharmed by the anti-bacterial composition.

### CROSS-REFERENCE TO RELATED APPLICATION

This application claims priority to a corresponding provisional application U.S. Serial No. 60/453,266, filed Mar. 7, 2003 in the name of the applicant of this application.

### FIELD OF THE INVENTION

This invention relates generally to anti-bacterial and sanitizing sprays and, more specifically, to an anti-bacterial spray composition for utensils and other objects, a portable aerosol-type dispenser therefor and method capable of allowing a user to use a portable, easy to carry aerosol-type device to sanitize or disinfect utensils.

### BACKGROUND OF THE INVENTION

According to the National Digestive Diseases Information Clearinghouse, harmful bacteria are the most common causes of foodborne illnesses. Harmful bacteria include Campylobacter jejuni (from raw and undercooked meat and poultry), L. monocytogenes, Salmonella, Shigella, Staphylococcus aureus, C. jejuni (from raw (unpasteurized) milk and dairy products, such as soft cheeses), Salmonella enteriditis (from raw or undercooked eggs), Vibrio vulnificus, Vibrio parahaemolyticus (from raw or undercooked shellfish) and C. botulinum (from improperly canned goods, and smoked or salted fish). In addition, produce such as lettuce, tomatoes, sprouts, and melons can become contaminated with Salmonella, Shigella, or Escherichia coli (E. coli) O157:H7. Contamination can occur during growing, harvesting, processing, storing, shipping, or final preparation. Sources of contamination are varied; however, these items are grown in the soil and therefore may become contaminated during growth or through processing and distribution. Contamination may also occur during food preparation in a restaurant or in a person's kitchen.

It is well-known that the conditions necessary for prevention of rapid bacterial growth are maintaining food outside the “safe temperature zones”, i.e. greater than 140° F. and less than 40° F., as well as keeping the environment surrounding food free of moisture, which are both conditions necessary for bacterial growth and replication. When food is cooked and left out for more than 2 hours at room temperature, bacteria can multiply quickly. Most bacteria grow undetected because they do not produce an “off” odor or change the color or texture of the food. Freezing food slows or stops bacteria's growth but does not destroy the bacteria. The microbes can become reactivated when the food is thawed. Refrigeration may slow the growth of some bacteria, but thorough cooking is needed to destroy the bacteria.

In most cases of foodborne illness, symptoms resemble intestinal flu and may last a few hours or even several days. Symptoms can range from mild to serious and include abdominal cramps, nausea, vomiting, diarrhea, fever and dehydration.

In most jurisdictions hygienic regulations required for eating establishments are regulated. Nevertheless, it is common for sloppiness to occur in the handling of utensils, particularly when an eating establishment becomes busy. In addition, there are many public areas where hygienic compliance does not exist.

Eating out has become popular for many people thereby increasing the risk of the occurrence of food-borne illness. Many people have a genuine concern that the utensils supplied in an eating establishment are incompletely sanitized. For example, it is not uncommon to see customers wiping their utensils before using them while visiting a restaurant. For many people, similar feelings exist regarding the sanitization of soda pop cans obtained from dispensers, and any item that may have common use by many people, such as eating surfaces in public places, sporting events, musical instruments such as keyboards and internet cafes.

Currently, apart from a reliance on people's good hygiene practices, no suitable method exists for insuring that eating out will be completely safe. The disclosure of this application addresses compositions and methods for providing for sanitization or disinfection of utensils and objects that will result in more hygienic conditions for the user of utensils and objects which could come into direct and/or indirect contact with a user's mouth.

### SUMMARY OF THE INVENTION

It is an object of the present invention to provide improved anti-bacterial compositions suitable for sanitizing and/or disinfecting utensils and other objects prior to coming into direct and/or indirect contact with a user's mouth.

It is a further object of the present invention to provide improved anti-bacterial compositions in the form of a spray that are suitable for sanitizing and/or disinfecting utensils and other objects prior to coming into direct and/or indirect contact with a user's mouth.

It is a still further object of the present invention to provide an improved portable aerosol-type dispenser suitable for sanitizing and/or disinfecting utensils and other objects prior to coming into direct and/or indirect contact with a user's mouth.

It is a further object of the present invention to provide improved anti-bacterial compositions in the form of a packaged wipe that are suitable for sanitizing and/or disinfecting utensils and other objects prior to coming into direct and/or indirect contact with a user's mouth.

It is a yet further object of the present invention to provide methods for using improved anti-bacterial compositions that are suitable for sanitizing and/or disinfecting utensils and other objects prior to coming into direct and/or indirect contact with a user's mouth.

### BRIEF DESCRIPTION OF THE PREFERRED EMBODIMENTS

In accordance with one embodiment of the present invention, an anti-bacterial utensil spray composition comprises, in combination, at least one anti-bacterial substantially non-residue-producing polar, protic solvent having a low molecular weight and a high level of volatility and having one of low toxicity and no toxicity, and at least one residue-producing anti-bacterial component having one of low toxicity and no toxicity, the at least one solvent and the at least one component providing both fast-acting and long-acting sanitization when applied to a surface of a contaminated object.

In accordance with a second embodiment of the present invention, a method for disinfecting utensils comprises the steps of providing a portable aerosol-type dispenser having an anti-bacterial composition having one of low toxicity and no toxicity and being capable of disinfecting utensils, and activating the aerosol-type dispenser to release a spray of the anti-bacterial composition onto utensils in order to disinfect them.

In accordance with a third embodiment of the present invention, a method for disinfecting utensils using an impregnated fabric comprises the steps of providing a sealed package comprising an impregnated fabric having an anti-bacterial composition capable of disinfecting utensils, providing the anti-bacterial composition comprises a combination of at least one non-toxic anti-bacterial substantially non-residue-producing polar, protic solvent having a low molecular weight and a high level of volatility and having one of low toxicity and no toxicity and at least one residue-producing anti-bacterial component having one of low toxicity and no toxicity, the at least one solvent and the at least one component providing both fast-acting and long-acting sanitization when applied to a surface of a contaminated object, unpacking the impregnated fabric from the sealed package, and wiping the utensils with the impregnated fabric to disinfect the utensils.

In accordance with a fourth embodiment of the present invention, a method for disinfecting objects comprising the steps of providing a portable aerosol-type dispenser including an anti-bacterial composition, the anti-bacterial composition comprising a combination of at least one anti-bacterial substantially non-residue-producing polar, protic solvent having a low molecular weight and a high level of volatility and having one of low toxicity and no toxicity and at least one residue-producing anti-bacterial component having one of low toxicity and no toxicity, the at least one solvent and the at least one component providing both a fast-acting and long-acting effect capable of disinfecting objects so that a user's mouth coming into both direct and indirect contact with the anti-bacterial composition is unharmed by the anti-bacterial composition, and activating the aerosol-type dispenser to release a spray of the anti-bacterial composition onto the objects in order to disinfect the objects.

### BRIEF DESCRIPTION OF THE DRAWINGS

FIG. 1 shows a chemical formula for an embodiment of a halophenolic antibacterial compound.

FIG. 2 is a side elevational view of a portable aerosol-type dispenser.

### DETAILED DESCRIPTION OF THE PREFERRED EMBODIMENTS

Various anti-bacterial compounds are available for use in the formulations of this disclosure. Anti-bacterial compounds may generally be divided into two groups: (1) non-residue-producing, characterized by quick disappearance by evaporation or breakdown, examples of which include alcohols, halogen releasing compounds, peroxides and aldehydes and; (2) residue-producing which are long-acting on the surface to be sanitized, examples of which include: anilides, biguanides, substituted phenols, bisphenols and halophenols and quaternary ammonium compounds and more specifically Triclosan, Triclocarban and benzalkonium chlorides and the like.

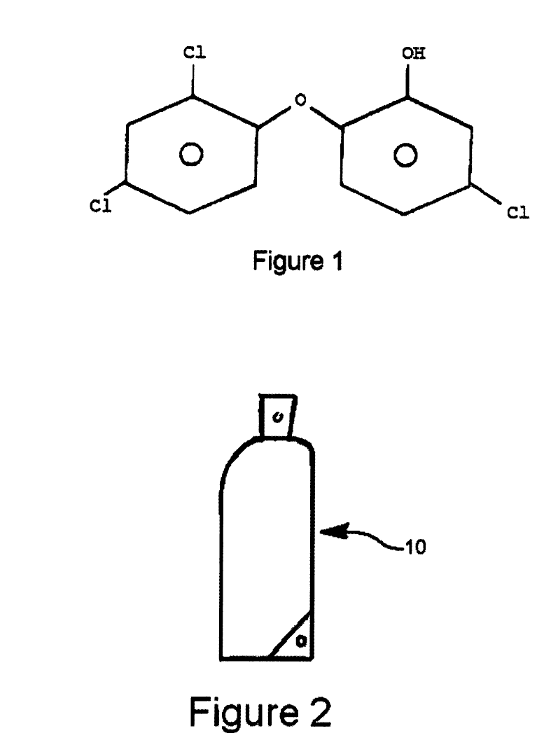

According to the above description, a preferred anti-bacterial utensil spray composition comprises, in combination at least one anti-bacterial substantially non-residue-producing polar, protic solvent having a low molecular weight and a high level of volatility and at least one residue-producing anti-bacterial component. Both the solvent and the component must either be non-toxic or have a low level of toxicity (for example, a level of toxicity that would qualify for a rating of GRAS (“Generally Regarded As Safe”) by the EPA or FDA). The preferred anti-bacterial utensil spray compositions of this disclosure consist essentially of about 60-99.9% concentration (wt) ethanol, about 0-40% concentration (wt) water and about 0.1-1% concentration (wt) Triclosan. It has been found to be advantageous to use a combination of residue-producing and non-residue-producing anti-bacterial compounds. In the preferred embodiment, the composition comprises about 0.3% concentration (wt) Triclosan and about 99.7% concentration (wt) of a polar, protic solvent having a low molecular weight and having a high level of volatility. A preferred polar, protic solvent is Ethanol, specifically denatured Ethanol. A further preferred polar, protic solvent is Anhydrol 200 manufactured by Union Carbide, CT, U.S.A. Anhydrol 200 comprises a mixture of about 86% concentration (wt) Ethanol, about 9% concentration (wt) Isopropanol, about 4% concentration (wt) Methanol and about 1% concentration (wt) Methyl Isobutyl Ketone. It is to be understood that a substantially non-toxic fragrant may also be added to the anti-bacterial utensil spray. A preferred useful substantially non-toxic fragrant is about 0.1% concentration (wt) Lemon Fragrance Agent (Lemon #YY78-01503). Other suitable formulations of the anti-bacterial spray comprise about 0.1% to about 0.5% concentration (wt) Triclosan, less than about 0.2% concentration (wt) Lemon Fragrance Agent (Lemon #YY78-01503) and the balance (i.e. about 99.3% to about 99.9% concentration (wt)) of Anhydrol 200. In the preferred embodiment, the Triclosan (CAS No: 3380-34-5) consists of the chemical formula: 2,4,4′-Trichloro-2′-hydroxydiphenyl ether (as shown in FIG. 1). Triclosan is available from numerous manufacturers, one example of a manufacturer being Ciba Specialty Chemicals, Inc.

In an alternative formulation the anti-bacterial spray consists essentially of approximately 0.3% concentration (wt) Triclosan, approximately 70% concentration (wt) Ethanol and approximately 29.7% concentration (wt) de-ionized water. The Ethanol is Specially Denatured Ethanol SD-40 (CAS# 64-17-5), although, it should be clearly understood that substantial benefit could be derived from another form of Ethanol and in a different percentage within the range of 60-99.9%, so long as it is capable of effectively killing bacteria. Preferably, the remainder of the composition is de-ionized water, although it should be clearly understood that substantial benefit can be derived from an alternative configuration of the composition, in which the water is not de-ionized, or in which the composition is anhydrous.

As described above, the ideal solvent is one that is polar, protic with a low molecular weight and a high level of volatility. Ethanol is the ideal solvent. Several denatured alcohol mixtures were tested and Anhydrol 200 was determined to be the best solvent. Specially Denatured Alcohol SD40 was determined to be less suitable because Special Denatured Alcohol SD40 evaporated more slowly.

The present invention further comprises a method for disinfecting utensils comprising the steps of providing a portable aerosol-type dispenser 10 (see FIG. 2) having an anti-bacterial composition capable of disinfecting utensils, and activating the aerosol-type dispenser to release a spray of the anti-bacterial composition onto utensils in order to disinfect them. It should be clearly understood that “aerosol-type” dispenser 10 includes any dispenser capable of releasing a liquid-type substance in a spray form, such as a perfume-type spray bottle, a pump-spray and the like. Preferably, the anti-bacterial composition comprises about 0.3% concentration (wt) Triclosan, about 0.1% concentration (wt) Lemon Fragrance Agent (Lemon #YY78-01503) and about 99.6% concentration (wt) Anhydrol 200. In an alternative formulation, the anti-bacterial spray consists essentially of approximately 0.3% concentration (wt) Triclosan, 70% concentration (wt) Ethanol and approximately 29.7% concentration (wt) de-ionized water. The Ethanol is Specially Denatured Ethanol SD-40 (CAS# 64-17-5), although, it should be clearly understood that substantial benefit could be derived from another form of Ethanol and in a different percentage within the range of 60-99.9%, so long as it is capable of effectively killing bacteria. It should be clearly understood that substantial benefit could be derived from an alternative anti-bacterial composition, so long as it is capable of disinfecting utensils or other objects in a substantially non-toxic or low-toxic manner so that a user could place the utensils or other objects in or around the user's mouth without the anti-bacterial composition harming the user.

In an alternative embodiment, a method for disinfecting utensils comprises the steps of providing a sealed package comprising an impregnated fabric having an anti-bacterial composition having one of low toxicity and no toxicity and being capable of disinfecting utensils, unpacking the impregnated fabric from the sealed package and, wiping the utensils with the impregnated fabric to disinfect the utensils. The impregnated fabric suitable for providing anti-bacterial activity comprises a woven or a non-woven synthetic fabric or a natural fabric such polyester, nylon, cotton and the like. The packaging comprises a plastic and can further comprise a metal lining such as aluminum or the like. Preferably, the anti-bacterial composition comprises about 0.3% concentration (wt) Triclosan, about 0.1% concentration (wt) Lemon Fragrance Agent (Lemon #YY78-01503) and about 99.6% concentration (wt) Anhydrol 200. In an alternative formulation the anti-bacterial spray consists essentially of approximately 0.3% concentration (wt) Triclosan, 70% concentration (wt) Ethanol and approximately 29.7% concentration (wt) de-ionized water. The Ethanol is Specially Denatured Ethanol SD-40 (CAS# 64-17-5), although, it should be clearly understood that substantial benefit could be derived from another form of Ethanol and in a different percentage within the range of 60-99.9%, so long as it is capable of effectively killing bacteria. It should be clearly understood that substantial benefit could be derived from an alternative non-toxic anti-bacterial composition, so long as it is capable of disinfecting utensils or other objects in a non-toxic manner so that a user could place the utensils or other objects in or around the user's mouth without the non-toxic anti-bacterial composition harming the user. An impregnated fabric as described above differs from known sanitizing fabrics because known sanitizing fabrics chemically bind the sanitizing agent to the fabric. In the current disclosure, the formulation comprises a combination of anti-bacterial compounds which are not chemically bound to the fabric and provide a combination of anti-bacterial non-residue-producing and residue-producing effects as discussed above.

The present invention further comprises a method for disinfecting objects comprising the steps of providing a portable aerosol-type dispenser having an anti-bacterial composition capable of disinfecting objects so that a user's mouth could come into direct and/or indirect contact with the anti-bacterial composition without the anti-bacterial composition harming the user, and activating the aerosol-type dispenser 10 to release a spray of the anti-bacterial composition onto objects in order to disinfect them. This method is capable of being used on any object which could come into direct and/or indirect contact with a user's mouth, such as a cup, a soda can, an eating surface, a keyboard, etc.

While, in the preferred embodiment, the method for disinfecting utensils comprises the portable aerosol-type dispenser 10, it should be clearly understood that any portable container capable of holding an anti-bacterial substance that can be dispensed under pressure, such as a hand-pumped spraying device, is within the spirit and scope of this invention. Preferably, the portable aerosol-type dispenser 10 is small enough to be placed in a shirt or pants pocket so that the portable aerosol-type dispenser 10 can easily be carried by a user in an unobtrusive manner. FIG. 2 illustrates an example of the portable aerosol-type dispenser 10. In a preferred embodiment the aerosol-type dispenser 10 comprises a canister comprising a metal such as aluminum and the like, an actuator comprising a plastic such as polypropylene and the like, a pump dispensing about 50 μl per use and a cap comprising a plastic such as polypropylene, high density polyethylene and the like. It is understood that the size of the canister can be varied according to need, and the plastic used for the actuator and cap can be any suitable polymer and copolymer that is moldable, such as polyester, nylon, polyvinyl chloride and the like. It is further understood that the amount of spray to be emitted by the aerosol-type dispenser 10 per use can be varied depending on regulatory requirements and that the canister can be made of metal, plastic, glass, a combination of these and other suitable materials. In addition, it is understood that the materials from which the aerosol-type dispenser 10 is constructed can be colored as required.

While the disclosure has been particularly shown and described with reference to preferred embodiments thereof, it will be understood by those skilled in the art that the foregoing and other changes in form and details may be made therein without departing from the spirit and scope of the disclosure. For example, the present invention may be used to disinfect a toilet seat area.

What is claimed is:

1. An anti-bacterial utensil spray composition comprising, in combination:
at least one anti-bacterial substantially non-residue-producing polar, protic solvent having a low molecular weight and a high level of volatility and having one of low toxicity and no toxicity; and
at least one residue-producing anti-bacterial component having one of low toxicity and no toxicity, said at least one solvent and said at least one component providing both fast-acting and long-acting sanitization when applied to a surface of a contaminated object.

2. The anti-bacterial utensil spray composition according to claim 1 further comprising a fragrant.

3. The anti-bacterial utensil spray composition according to claim 1 wherein said anti-bacterial substantially non-residue-producing polar, protic solvent having a low molecular weight and a high level of volatility and having one of low toxicity and no toxicity is selected from the group consisting of Ethanol, Special Denatured Alcohol SD-40, and Anhydrol 200.

4. The anti-bacterial utensil spray composition according to claim 1 wherein said residue-producing anti-bacterial component having one of low toxicity and no toxicity comprises Triclosan.

5. The anti-bacterial utensil spray composition according to claim 1 consisting essentially of about 60-99.9% concentration (wt) Ethanol, about 0-40% concentration (wt) water and about 0.1-1% concentration (wt) Triclosan.

6. The anti-bacterial utensil spray composition according to claim 1 comprising about 0.1% to about 1% concentration (wt) Triclosan, less than about 0.2% concentration (wt) Lemon Fragrance Agent (Lemon #YY78-01503) and about 98.8% to about 99.9% concentration (wt) Anhydrol 200.

7. The anti-bacterial utensil spray composition according to claim 1 comprising about 0.3% concentration (wt) Triclosan and about 99.7% concentration (wt) Anhydrol 200.

8. The anti-bacterial utensil spray composition according to claim 7 further comprising about 0.1% concentration (wt) Lemon Fragrance Agent (Lemon #YY78-01503).

9. The anti-bacterial utensil spray composition according to claim 1 consisting essentially of approximately 0.3% concentration (wt) Triclosan, approximately 70% concentration (wt) Ethanol and approximately 29.7% concentration (wt) de-ionized water.

10. The anti-bacterial utensil spray composition according to claim 9 wherein said Ethanol is denatured.

11. A method for disinfecting utensils comprising the steps of:
providing a portable aerosol-type dispenser having an anti-bacterial composition having one of low toxicity and no toxicity and being capable of disinfecting utensils; and
activating the aerosol-type dispenser to release a spray of said anti-bacterial composition onto utensils in order to disinfect them.

12. The method for disinfecting utensils according to claim 11 further comprising the step of: providing said anti-bacterial composition comprising a combination of an anti-bacterial substantially non-residue-producing polar, protic solvent having a low molecular weight and a high level of volatility and having one of low toxicity and no toxicity and a residue-producing anti-bacterial component having one of low toxicity and no toxicity, said solvent and said component providing both fast-acting and long-acting sanitization when applied to a surface of a contaminated object.

13. The method for disinfecting utensils according to claim 11 further comprising the step of: providing said anti-bacterial composition comprising about 0.3% concentration (wt) Triclosan, about 0.1% concentration (wt) Lemon Fragrance Agent (Lemon #YY78-01503) and about 99.6% concentration (wt) Anhydrol 200.

14. The method for disinfecting utensils according to claim 11 further comprising the step of: providing said anti-bacterial composition consisting essentially of approximately 0.3% concentration (wt) Triclosan, 70% concentration (wt) Ethanol and approximately 29.7% concentration (wt) de-ionized water.

15. A method for disinfecting utensils using an impregnated fabric comprising the steps of:
providing a sealed package comprising an impregnated fabric having an anti-bacterial composition capable of disinfecting utensils;
providing said anti-bacterial composition comprises a combination of at least one anti-bacterial substantially non-residue-producing polar, protic solvent having a low molecular weight and a high level of volatility and having one of low toxicity and no toxicity and at least one residue-producing anti-bacterial component having one of low toxicity and no toxicity, said at least one solvent and said at least one component providing both fast-acting and long-acting sanitization when applied to a surface of a contaminated object;
unpacking said impregnated fabric from said sealed package; and
wiping the utensils with said impregnated fabric to disinfect the utensils.

16. The method for disinfecting utensils using an impregnated fabric according to claim 15 further comprising the step of: providing said anti-bacterial composition comprising about 0.3% concentration (wt) Triclosan, about 0.1% concentration (wt) Lemon Fragrance Agent (Lemon #YY78-01503) and about 99.6% concentration (wt) Anhydrol 200.

17. The method for disinfecting utensils using an impregnated fabric according to claim 15 further comprising the step of: providing said anti-bacterial composition consisting essentially of approximately 0.3% concentration (wt) Triclosan, 70% concentration (wt) Ethanol and approximately 29.7% concentration (wt) de-ionized water.

18. A method for disinfecting objects comprising the steps of:
providing a portable aerosol-type dispenser including a anti-bacterial composition, said anti-bacterial composition comprising a combination of at least one anti-bacterial substantially non-residue-producing polar, protic solvent having a low molecular weight and a high level of volatility and having one of low toxicity and no toxicity and at least one residue-producing anti-bacterial component having one of low toxicity and no toxicity, said at least one solvent and said at least one component providing both a fast-acting and long-acting effect capable of disinfecting objects so that a user's mouth coming into both direct and indirect contact with said anti-bacterial composition is unharmed by said anti-bacterial composition; and
activating the aerosol-type dispenser to release a spray of said anti-bacterial composition onto said objects in order to disinfect said objects.

19. The method for disinfecting objects according to claim 18 further comprising the step of: providing said anti-bacterial composition comprising about 0.3% concentration (wt) Triclosan, about 0.1% concentration (wt) Lemon Fragrance Agent (Lemon #YY78-01503) and about 99.6% concentration (wt) Anhydrol 200.

20. The method for disinfecting objects according to claim 18 further comprising the step of: providing said anti-bacterial composition consisting essentially of approximately 0.3% concentration (wt) Triclosan, 70% concentration (wt) Ethanol and approximately 29.7% concentration (wt) de-ionized water.
